(19) United States
(12) Patent Application Publication
(10) Pub. No.: US 2022/0177529 A1
(43) Pub. Date: Jun. 9, 2022

(54) FUSION PROTEIN FOR ENHANCING GENE EDITING AND USE THEREOF

(71) Applicant: BIORAY LABORATORIES INC., Shanghai (CN)

(72) Inventors: Dali LI, Shanghai (CN); Shuming YIN, Shanghai (CN); Mei ZHANG, Shanghai (CN); Xi CHEN, Shanghai (CN); Xiaohui ZHANG, Shanghai (CN); Liren WANG, Shanghai (CN); Mingyao LIU, Shanghai (CN)

(73) Assignee: BIORAY LABORATORIES INC., Shanghai (CN)

(21) Appl. No.: 17/440,777

(22) PCT Filed: Mar. 18, 2020
(86) PCT No.: PCT/CN2020/080032

§ 371 Date: Sep. 19, 2021

(30) Foreign Application Priority Data
Mar. 19, 2019 (CN) 201910210107.1

(51) Int. Cl. C07K 14/435; C12N 15/11; C12N 15/90; C12N 9/22; C12N 9/78; A61K 48/00

(52) U.S. Cl. CPC C07K 14/43581; C12N 15/111; C12N 15/90; C12N 9/22; C12N 2310/20; A61K 48/00; C12Y 305/04002; C12Y 305/04004; C07K 2319/80; C12N 9/78

(57) ABSTRACT

The present invention relates to a fusion protein for enhancing gene editing and use thereof. In particular, the invention provides an enhanced fusion protein. The enhanced fusion proteins of the present invention can significantly increase gene editing efficiency in vivo or in vitro as compared to the wildtype gene editing protein.

### TECHNICAL FIELD

The present invention relates to the field of biotechnology, in particular to a fusion protein for enhancing gene editing and its application.

### BACKGROUND

Gene editing is a genetic manipulation technology which modified genes by artificially achieving double-stranded DNA breaks and using the repair mechanism of double stranded DNA breaks. Current gene editing technologies include ZFN, TALEN and CRISPR/Cas9, among which CRISPR/Cas9 is the most widely applicability. CRISPR/Csa9 technology is a adaptive immune mechanism derived from bacteria or archaebacteria that uses a single-stranded guide RNA (sgRNA) and Cas9 protein to produce DNA double strand breaks at specific locations in the genome, then through endogenous non-homologous end binding (NHEJ) or homologous recombination (HDR) repair mechanism, the knockout of a target gene or the insertion of a specific gene or segment can be achieved.

Although the CRISPR/Cas9 technology is incredibly powerful, it also has some drawbacks, such as, 1. the concerns about off-target problem; 2. the limitation of PAM leads to the limitation of target site selection; 3. some newly developed tools (XCas9 and SpCas9-NG) show generally low editing efficiency. However, the current optimization and modification of gene editing tools are mainly based on the improvement of the accuracy and targeting scope of the tools, and there are no methods to comprehensively improve various gene editing tools for the gene editing tools themselves.

Therefore, there is an urgent need in this field to develop a new method to improve the efficiency of gene editing.

### SUMMARY OF THE INVENTION

The purpose of the present invention is to provide a new method for improving gene editing efficiency.

In a first aspect of the present invention, it provides a fusion protein, the structure of the fusion protein is shown in the following Formula I or I′:

C-A-L-B (I)

B-L-A-C (I′)

wherein

A is a gene editing protein,

B is a DNA double-strand binding domain,

C is an optional base editor element;

L is none or a linker peptide;

each “-” is independently a linker peptide or a peptide bond or a non-peptide bond.

In another preferred embodiment, when the structure of the fusion protein is as shown in formula I′, C is none.

In another preferred embodiment, the non-peptide bond includes PEG.

In another preferred embodiment, the gene editing protein is selected from the group consisting of Cas9, Cas12, Cas12a, Cas12b, Cas13, Cas14, and a combination thereof.

In another preferred embodiment, the gene editing protein includes wild type or mutant gene editing protein.

In another preferred embodiment, the gene editing protein is selected from the group consisting of Streptococcus pyogenes, Staphylococcus aureus, Acidaminococcus sp, Lachnospiraceae bacterium, and a combination thereof.

In another preferred embodiment, the amino acid sequence of the wild-type gene editing protein is shown in SEQ ID NO.: 1, 14 or 15.

In another preferred embodiment, the amino acid sequence of the base editor element is shown in SEQ ID NO.: 2 or 12.

In another preferred embodiment, the DNA double-strand binding domain is a non-sequence-specific DNA double-strand binding domain.

In another preferred embodiment, the DNA double-stranded binding domain is selected from the group consisting of HMG-D, Sac7d, and a combination thereof.

In another preferred embodiment, the DNA double-stranded binding domain includes a wild-type DNA double-stranded binding domain and a mutant DNA double-stranded binding domain.

In another preferred embodiment, the DNA double-stranded binding domain is derived from Drosophila or archaebacteria.

In another preferred embodiment, the amino acid sequence of the DNA double-strand binding domain is shown in SEQ ID NO.: 10 or 11.

In another preferred embodiment, the length of the linker peptide is 1-100 aa, preferably, 15-85 aa, more preferably, 25-70 aa.

In another preferred embodiment, the linker peptide is a sequence shown as Gly-Gly-Ser with n repeats, wherein n is 2-8, preferably n is 3-6.

In another preferred embodiment, the amino acid sequence of the linker peptide is selected from the group consisting of:

(1) a polypeptide having an amino acid sequence as shown in any one of SEQ ID NO.: 3-7; or

(2) a polypeptide formed by substituting, deleting or adding one or several, preferably 1-20, more preferably 1-15, more preferably 1-10, more preferably 1-8, more preferably 1-3, most preferably 1 amino acid residues to the amino acid sequence as shown in any one of SEQ ID NO.: 3-7, derived from a polypeptide having the amino acid sequence as shown in any one of SEQ ID NO.: 3-7 having the function of the polypeptide as described in (1).

In another preferred embodiment, the base editor element includes cytosine deaminase and adenine deaminase.

In another preferred embodiment, the cytosine deaminase includes Apobec1 and Apobec3A.

In another preferred embodiment, the adenine deaminase includes TadA.

In another preferred embodiment, the fusion protein has an amino acid sequence as shown in any one of SEQ ID NO.: 8, 9, and 13.

In a second aspect of the present invention, it provides a polynucleotide, which encodes the fusion protein according to the first aspect of the present invention.

In another preferred embodiment, the polynucleotide additionally contains an auxiliary element selected from the group consisting of signal peptide, secretory peptide, tag sequence (such as 6His), or a combination thereof on the flanking of the ORF of the mutant protein or fusion protein.

In another preferred embodiment, the polynucleotide is selected from the group consisting of: DNA sequence, RNA sequence, and a combination thereof.

In a third aspect of the present invention, it provides a vector containing the polynucleotide according to the second aspect of the present invention.

In another preferred embodiment, the vector includes one or more promoters, which are operably linked to the nucleic acid sequence, enhancer, transcription termination signal, polyadenylation sequence, origin of replication, selectable marker, nucleic acid restriction site, and/or homologous recombination site.

In another preferred embodiment, the vector includes a plasmid and viral vector.

In another preferred embodiment, the viral vector is selected from the group consisting of adeno-associated virus (AAV), adenovirus, lentivirus, retrovirus, herpes virus, SV40, poxvirus, and a combination thereof.

In another preferred embodiment, the vector includes an expression vector, a shuttle vector, and an integration vector.

In a fourth aspect of the present invention, it provides a host cell containing the vector according to the third aspect of the present invention, or its genome integrates the polynucleotide according to the second aspect of the present invention.

In another preferred embodiment, the host cell is a eukaryotic cell, such as a yeast cell, a plant cell or a mammalian cell (including a human and non-human mammal).

In another preferred embodiment, the host cell is a prokaryotic cell, such as Escherichia coli.

In another preferred embodiment, the yeast cell is selected from one or more yeasts from the group consisting of: Pichia pastoris, Kluyveromyces, and a combination thereof; preferably, the yeast cell includes: Kluyveromyces, more preferably Kluyveromyces marxianus, and/or Kluyveromyces lactis.

In another preferred embodiment, the host cell is selected from the group consisting of Escherichia coli, wheat germ cell, insect cell, SF9, Hela, HEK293, CHO, yeast cell, and a combination thereof.

In a fifth aspect of the present invention, it provides a method for producing the fusion protein according to the first aspect of the present invention, comprising the steps:

Under conditions suitable for expression, culturing the host cell according to the fourth aspect of the present invention, thereby expressing the fusion protein; and/or

Isolating the fusion protein.

In a sixth aspect of the present invention, it provides a gene editing reagent, which comprises the fusion protein according to the first aspect of the present invention.

In another preferred embodiment, the reagent further includes one or more reagents selected from the group consisting of:

(a1) gRNA, crRNA, or a vector for producing the gRNA or crRNA;

(a2) a template for homology-directed repair: single-stranded nucleotide sequence or plasmid vector.

In a seventh aspect of the present invention, it provides a kit comprising the gene editing reagent according to the sixth aspect of the present invention.

In another preferred embodiment, the kit further includes one or more reagents selected from group consisting of:

(a1) gRNA, crRNA, or a vector for producing the gRNA or crRNA;

(a2) a template for homology-directed repair: single-stranded nucleotide sequence or plasmid vector.

In another preferred embodiment, the kit further includes a label or instructions.

In an eighth aspect of the present invention, it provides a use of the fusion protein according to the first aspect of the present invention for the preparation of a reagent or kit for improving gene editing efficiency.

In a ninth aspect of the present invention, it provides a pharmaceutical composition, comprising:

(a) a fusion protein according to the first aspect of the present invention, or a coding gene thereof, or an expression vector (vector) thereof; and

(b) a pharmaceutically acceptable carrier (carrier).

In another preferred embodiment, the expression vector includes a viral vector.

In another preferred embodiment, the viral vector is selected from the group consisting of adeno-associated virus (AAV), adenovirus, lentivirus, retrovirus, herpes virus, SV40, poxvirus, and a combination thereof.

In another preferred embodiment, the vector is selected from the group consisting of: lentivirus, adenovirus, adeno-associated virus (AAV), and a combination thereof, preferably, the vector is adeno-associated virus (AAV).

In another preferred embodiment, the dosage form of the pharmaceutical composition is selected from the group consisting of a lyophilized preparation, a liquid preparation, and a combination thereof.

In another preferred embodiment, the dosage form of the pharmaceutical composition is an injection dosage form.

In another preferred embodiment, the pharmaceutical composition also includes other drugs for gene therapy.

In another preferred embodiment, the other drugs for gene therapy are selected from the group consisting of antisense nucleotide drugs, EDIT-101 drugs, CTX001, and a combination thereof.

In another preferred embodiment, the pharmaceutical composition is a cell preparation.

In a tenth aspect of the present invention, it provides a medicine kit comprising:

(a1) a first container, and the fusion protein according to the first aspect of the present invention, a coding gene thereof, or an expression vector thereof, or a medicine containing the fusion protein according to the first aspect of the present invention located in the first container.

In another preferred embodiment, the medicine kit further includes:

(a2) a second container, and other medicines for gene therapy, or medicines containing other medicines for gene therapy located in the second container.

In another preferred embodiment, the first container and the second container are the same or different containers.

In another preferred embodiment, the medicine located in the first container is a prescribed preparation containing the fusion protein of claim 1.

In another preferred embodiment, the medicine located in the second container is a prescribed preparation containing other medicines for gene therapy.

In another preferred embodiment, the dosage form of the medicine is selected from the group consisting of a lyophilized preparation, a liquid preparation, and a combination thereof.

In another preferred embodiment, the dosage form of the medicine is an injection dosage form.

In an eleventh aspect of the present invention, it provides a use of the fusion protein according to the first aspect of the present invention for the preparation of a medicine for gene therapy.

In a twelfth aspect of the present invention, it provides a method for improving gene editing efficiency, comprising the steps:

In the presence of the fusion protein according to the first aspect of the present invention or the gene editing reagent according to the sixth aspect of the present invention, performing gene editing on a cell, thereby improving the efficiency of gene editing.

In another preferred embodiment, the cell includes a human or non-human mammalian cell (such as a primate or livestock).

In another preferred embodiment, the cell includes a cancer cell or normal cell.

In another preferred embodiment, the cell is selected from the group consisting of a kidney cell, liver cell, nerve cell, heart cell, epithelial cell, muscle cell, somatic cell, bone marrow cell, endothelial cell, and a combination thereof.

In another preferred embodiment, the cell is selected from the group consisting of a 293 cell, A549 cell, SW626 cell, HT-3 cell, PA-1 cell, and a combination thereof.

In another preferred embodiment, the cell includes HEK293T.

In another preferred embodiment, the gene editing is performed in an in vitro reaction system.

In another preferred embodiment, in the in vitro reaction system, the content of the fusion protein or gene editing reagent is 100 ng-700 ng, preferably, 200 ng-600 ng, more preferably, 300 ng-500 ng.

In another preferred embodiment, the method is non-diagnostic and non-therapeutic.

In another preferred embodiment, the cell is an in vitro cell.

It should be understood that, within the scope of the present invention, the technical features specifically described above and below (such as the Examples) can be combined with each other, thereby constituting a new or preferred technical solution which needs not be described one by one.

### DESCRIPTION OF DRAWINGS

FIG. 1 shows the editing efficiency of two different endogenous targets, and it shows that the different fusion architecture between the DNA double-stranded binding domain and Cas9 protein, as well as the various linker length, have a difference effects on efficiency improvement. Comprehensively, we choose the best fusion architecture that the HMG-D domain fused to the N-terminal of Cas9 via L4 length linker, i.e., HMG-D-L4-Cas9, wherein H represents HMG-D, S represents Sac7d, L1-L5 represents various length linker, mutH represents mutant HMG-D (V32A and T33A mutations reduce the binding activity), C represents Cas9.

FIG. 2 shows HMG-D-L4-Cas9 can improve gene editing efficiency in the other endogenous target sites, and the efficiency improvement >20%, preferably, >40%, more preferably, >60% (such as 80%), up to 2 times, wherein H represents HMG-D.

FIG. 3 shows the double-strand binding domain HMG-D can improve the efficiency of Cas9 protein from other sources, like SaCas9, by more than 20%.

FIG. 4 shows the double-strand binding domain HMG-D can improve the efficiency of non-Cas9 proteins (such as AsCas12a) by 10-20%.

FIG. 5 shows the double-strand binding domain HMG-D can improve the efficiency of the epigenetic regulation tools (e.g., CRISPR-VPR), the efficiency can be increased by 2 times and wherein endCas9 is HMG-D-L4-dCas9.

FIG. 6 shows the double-strand binding domain HMG-D can improve the efficiency of the single-base editing tool ABE, wherein H represents HMG-D.

### DETAILED DESCRIPTION

After extensive and in-depth research, the inventors have unexpectedly obtained an enhanced fusion protein. Compared with wild-type gene editing protein, the enhanced fusion protein of the present invention can significantly improve gene editing efficiency in vivo or in vitro, and the present invention has also unexpectedly discovered that fusion protein formed by gene editing protein and DNA double-strand binding domain, optional base editor element and optional linker peptide can significantly improve gene editing efficiency (increased by ≥20%, such as 80%, or even up to 2 times). In addition, the present invention has also unexpectedly discovered that the fusion protein of the present invention can be used in gene therapy. On this basis, the inventors have completed the present invention.

### Terms

In order to make it easier to understand the present disclosure, first defining certain terms. As used in this application, unless expressly stated otherwise herein, each of the following terms shall have the meaning given below. Other definitions are stated throughout the application.

The term “about” may refer to a value or composition within an acceptable error range of a particular value or composition determined by a person of ordinary skill in the art, which will depend in part on how the value or composition is measured or determined. For example, as used herein, the expression “about 100” includes all values between 99 and 101 (e.g., 99.1, 99.2, 99.3, 99.4, etc.).

As used herein, the term “containing” or “comprising (including)” can be open, semi-closed, and closed. In other words, the term also includes “substantially consisting of” or “consisting of”.

Sequence identity (or homology) is determined by comparing two aligned sequences along a predetermined comparison window (it can be 50%, 60%, 70%, 80%, 90%, 95% or 100% of the length of the reference nucleotide sequence or protein) and determining the number of positions where the same residue appears. Usually, this is expressed as a percentage. The measurement of sequence identity of nucleotide sequences is a method well known to those skilled in the art.

As used herein, the term “EDIT-101 drug” belongs to gene therapy drugs, which is a type of cell. Specifically, EDIT-101 is a drug that uses CRISPR gene editing technology to treat hereditary retinal degeneration disease (LCA10 disease), EDIT-101 is administered by subretinal injection, and the gene editing system is directly delivered to the photoreceptor cells to achieve the therapeutic effect.

As used herein, the term “CTX001” belongs to gene therapy drugs, which is a type of cell. Specifically, CTX001 is based on CRISPR gene editing technology to achieve therapeutic purposes by cutting the BCL11A gene of patients with β-thalassemia.

Wild-Type Gene Editing Protein

As used herein, “wild-type gene editing protein” refers to a naturally occurring gene editing protein that has not been artificially modified. Its nucleotides can be obtained through genetic engineering techniques, such as genome sequencing, polymerase chain reaction (PCR) etc. The amino acid sequence can be deduced from the nucleotide sequence. The source of the wild-type gene editing protein includes (but is not limited to): Streptococcus pyogenes, Staphylococcus aureus, Acidaminococcus sp, Lachnospiraceae bacterium.

In a preferred embodiment of the present invention, the amino acid sequence of the wild-type gene editing protein is shown in SEQ ID NO.: 1 or 14 or 15.

In a preferred embodiment of the present invention, the gene editing protein includes, but is not limited to, Cas9, Cas9a, Cas12, Cas12a, Cas12b, Cas13, and Cas14.

DNA Double-Strand Binding Domain

As used herein, the term “DNA double-strand binding domain” is a DNA double-strand binding domain without sequence specificity. Compared with the sequence-specific DNA double-stranded binding domain, the non-sequence-specific DNA double-stranded binding domain of the present invention is not limited by the DNA sequence, and theoretically can bind to any DNA sequence. Therefore, it can be applied to any position of DNA binding. A preferred sequence of DNA double-strand binding domain is shown in SEQ ID NO.: 10 or 11.

Base Editor

Any base editor provided herein can modify specific nucleotide bases without generating a significant proportion of insertion/deletion. As used herein, “insertion/deletion” refers to the insertion or deletion of nucleotide bases within a nucleic acid. Such insertions or deletions can lead to frameshift mutations in the coding region of the gene. In some embodiments, it is desirable to produce a base editor that effectively modifies (e.g., mutates or deamination) specific nucleotides within a nucleic acid without generating a large number of insertions or deletions (i.e., insertion/deletion) in the nucleic acid. In certain embodiments, any of the base editors provided herein are capable of producing a larger proportion of the intended modification (e.g., point mutations or deamination) relative to insertion/deletion.

Any base editor of the present invention can effectively generate intended mutations, such as point mutations, in nucleic acids (for example, nucleic acids in the genome) without generating a large number of unintended mutations, such as unintended point mutations.

In the present invention, the base editor includes cytosine deaminase and adenine deaminase, and other types of base editors as long as they have the functions of the base editor of the present invention are also within the protection scope of the present invention.

In the present invention, the structure after the gene editing protein is fused with the base editor is called ABE or CBE, wherein ABE is the structure of gene editing protein fused with adenine deaminase and CBE is the structure of gene editing protein fused with cytosine deaminase.

The sequence of a preferred base editor is shown in SEQ ID NO.: 2 or 12.

Fusion Protein

As used herein, “fusion protein of the present invention” or “polypeptide” refers to the fusion protein according to the second aspect of the present invention. The structure of the fusion protein of the present invention is shown in the following Formula I or I′:

C-A-L-B (I)

B-L-A-C (I′)

wherein

A is a gene editing protein,

B is a DNA double-strand binding domain,

C is an optional base editor element;

L is none or a linker peptide,

each “-” is independently a linker peptide or a peptide bond or a non-peptide bond.

In the present invention, the length of the linker peptide has an effect on the activity of the fusion protein. The preferred length of the linker peptide is 1-100 aa, preferably, 15-85 aa, and more preferably, 25-70 aa.

A preferred linker peptide is shown in SEQ ID NO.: 3-7.

As used herein, the term “fusion protein” also includes variant forms as shown in SEQ ID NO.: 8, 9, or 13 having the above-mentioned activity. These variant forms include (but are not limited to): 1-3 (usually 1-2, more preferably 1) amino acid deletions, insertions and/or substitutions, and adding or deleting one or several (usually within 3, preferably within 2, more preferably within 1) amino acid at the C-terminal and/or N-terminal. For example, in this field, when amino acids with close or similar properties are substituted, the function of the protein is usually not changed. For another example, adding or deleting one or several amino acids at the C-terminus and/or N-terminus usually does not change the structure and function of the protein. In addition, the term also includes the polypeptide of the present invention in monomeric and multimeric forms. The term also includes linear and non-linear polypeptides (such as cyclic peptides).

The present invention also includes active fragments, derivatives and analogs of the above-mentioned fusion protein. As used herein, the terms “fragment”, “derivative” and “analog” refer to a polypeptide that substantially retains the function or activity of the fusion protein of the present invention. The polypeptide fragments, derivatives or analogues of the present invention can be (i) a polypeptide in which one or more conservative or non-conservative amino acid residues (preferably conservative amino acid residues) are substituted, or (ii) a polypeptide with a substitution group in one or more amino acid residues, or (iii) a polypeptide formed by fusion of an antigen peptide with another compound (such as a compound that prolongs the half-life of the polypeptide, such as polyethylene glycol), or (iv) the polypeptide formed by fusion of additional amino acid sequence to this polypeptide sequence (fusion protein formed by fusion with leader sequence, secretory sequence or 6His tag sequence). According to the teachings herein, these fragments, derivatives and analogs fall within the scope of those skilled in the art.

A preferred type of active derivative means that compared with the amino acid sequence of Formula I, there are at most 3, preferably at most 2, and more preferably at most 1 amino acid replaced by an amino acid with close or similar properties to form a polypeptide. These conservative variant polypeptides are best produced according to Table A by performing amino acid substitutions.

TABLE A

| Initial | Representative | Preferred |
|---|---|---|
| residues | substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The present invention also provides analogs of the fusion protein of the present invention. The difference between these analogs and the polypeptide as shown in SEQ ID NO.: 8 or 9 or 13 may be a difference in amino acid sequence, may also be a difference in modified form that does not affect the sequence, or both. Analogs also include analogs having residues different from natural L-amino acids (such as D-amino acids), and analogs having non-naturally occurring or synthetic amino acids (such as β, γ-amino acids). It should be understood that the polypeptide of the present invention is not limited to the representative polypeptides exemplified above.

Modified (usually without changing the primary structure) forms include: chemically derived forms of polypeptides in vivo or in vitro, such as acetylation or carboxylation. Modifications also include glycosylation, such as those polypeptides produced by glycosylation modifications during the synthesis and processing of the polypeptide or during further processing steps. This modification can be accomplished by exposing the polypeptide to an enzyme that performs glycosylation (such as a mammalian glycosylase or deglycosylase). Modified forms also include sequences with phosphorylated amino acid residues (such as phosphotyrosine, phosphoserine, phosphothreonine). It also includes polypeptides that have been modified to improve their anti-proteolytic properties or optimize their solubility properties.

In the present invention, in Formula I, A is a gene editing protein, B is HMG-D or Sac7d, C is adenine deaminase or cytosine deaminase or none, and L is L1 or L2 or L3 or L4 or L5 or none.

In a preferred embodiment, in Formula I, A is a gene editing protein, B is HMG-D, C is either adenine deaminase or cytosine deaminase or none, and L is L4 or L5.

In a preferred embodiment, the fusion protein of the present invention may also include two or more of the A, B, C, and L elements in Formula I.

In a preferred embodiment, in Formula I, A is a gene editing protein, B is HMG-9, C is none, and L is L4.

In a preferred embodiment, in Formula I, A is a gene editing protein, B is HMG-D, C is adenine deaminase, and L is L5.

In a preferred embodiment, in Formula I, A is a gene editing protein, B is HMG-D, C is cytosine deaminase, and L is L5.

In a preferred embodiment, the amino acid sequence of the fusion protein of the present invention is as shown in SEQ ID NO.: 8, 9 or 13.

Adeno-Associated Virus

Because Adeno-associated virus (AAV) is smaller than other viral vectors, and is non-pathogenic, and can be transfected into dividing and undivided cells, gene therapy methods for genetic diseases based on AAV vectors have received widespread attention.

Adeno-associated virus (AAV), also known as adeno associated virus, belongs to the family Parvoviridae and the genus Dependovirus. It is the simplest single-stranded DNA-deficient virus found so far, and requires helper viruses (usually adenoviruses) to participate in replication. It encodes cap and rep genes in the inverted repeat sequence (ITR) at both ends. ITRs play a decisive role in virus replication and packaging. Cap gene encodes virus capsid protein, and rep gene participates in virus replication and integration. AAV can infect a variety of cells.

Recombinant adeno-associated virus vector (rAAV) is derived from non-pathogenic wild-type adeno-associated virus. Due to its good safety, wide range of host cells (divided and non-divided cells), low immunogenicity, and long time to express foreign genes in vivo, it is regarded as one of the most promising gene transfer vectors and has been widely used in gene therapy and vaccine research worldwide. After more than 10 years of research, the biological characteristics of recombinant adeno-associated virus have been deeply understood, especially in terms of its application effects in various cells, tissues and in vivo experiments, in which a lot of information have been accumulated. In medical research, rAAV is used in gene therapy for various diseases (including in vivo and in vitro experiments). At the same time, as a characteristic gene transfer vector, it is also widely used in gene function research, disease model construction, gene knockout mouse preparation, etc.

In a preferred embodiment of the present invention, the vector is a recombinant AAV vector. AAVs are relatively small DNA viruses that can be integrated into the genome of the cells they infect in a stable and site-specific manner. They can infect a wide range of cells without any effect on cell growth, morphology or differentiation, and they do not seem to be involved in human pathology. AAV genome has been cloned, sequenced and characterized. AAV contains inverted terminal repeat (ITR) regions of about 145 bases at each end, which serve as the origin of replication of the virus. The rest of the genome is divided into two important regions with encapsidation functions: the left portion of the genome containing the rep gene involved in viral replication and viral gene expression; and the right portion of the genome comprising the cap gene encoding the viral capsid protein.

AAV vectors can be prepared using standard methods in the art. Any serotype of adeno-associated virus is suitable. Methods for purifying vectors can be found, for example, in U.S. Pat. Nos. 6,566,118, 6,989,264 and 6,995,006, the disclosures of which are incorporated herein by reference in their entirety. The preparation of hybrid vectors is described, for example, in PCT application No. PCT/US2005/027091, the disclosure of which is incorporated herein by reference in its entirety. The use of AAV-derived vectors for transporting genes in vitro and in vivo has been described (see, for example, International Patent Application Publication Nos. WO 91/18088 and WO 93/09239; U.S. Pat. Nos. 4,797,368, 6,596,535 and 5,139,941, and European Pat. No. 0488,528, which are incorporated herein by reference in their entirety). These patent publications describe various constructs derived from AAV in which the rep and/or cap genes are deleted and replaced by the gene of interest, and the use of these constructs to transport the gene of interest in vitro (into cultured cells) or in vivo (directly into organisms). Replication-deficient recombinant AAV can be prepared by co-transfecting the following plasmids into cell lines infected by human helper viruses (such as adenoviruses): plasmids containing nucleic acid sequences of interest flanked by two AAV inverted terminal repeat (ITR) regions, and plasmids carrying AAV capsizing genes (rep and cap genes). The resulting AAV recombinant was then purified by standard techniques.

In some embodiments, the recombinant vector is capsized into viral particles (for example, AAV virus particles including but not limited to AAV1, AAV2, AAV3, AAV4, AAV5, AAV6, AAV7, AAV8, AAV9, AAV10, AAV11, AAV12, AAV13, AAV14, AAV15 and AAV16). Accordingly, the present disclosure includes recombinant viral particles (recombinant because they contain recombinant polynucleotides) comprising any of the vectors described herein. Methods of producing such particles are known in the art and are described in U.S. Pat. No. 6,596,535.

Expression Vectors and Host Cells

The invention also relates to a vector containing the polynucleotide of the present invention and a host cell produced by genetic engineering using the vector of the present invention or the coding sequence of the fusion protein of the present invention, and a method for producing the polypeptide of the present invention by recombinant technology.

Through conventional recombinant DNA technology, the polynucleotide sequence of the present invention can be used to express or produce recombinant fusion protein. Generally speaking, there are the following steps:

(1) using the polynucleotide (or variant) of the present invention encoding the fusion protein of the present invention, or using a recombinant expression vector containing the polynucleotide to transform or transduce a suitable host cell;

(2). a host cell cultured in a suitable medium;

(3). separating and purifying protein from culture medium or cells.

In the present invention, the polynucleotide sequence encoding the fusion protein can be inserted into a recombinant expression vector. The term “recombinant expression vector” refers to bacterial plasmids, bacteriophages, yeast plasmids, plant cell viruses, mammalian cell viruses such as adenovirus, retrovirus or other vectors well known in the art. Any plasmid and vector can be used as long as it can be replicated and stabilized in the host. An important feature of an expression vector is that it usually contains an origin of replication, a promoter, a marker gene, and translation control elements.

Methods well known to those skilled in the art can be used to construct an expression vector containing the DNA sequence encoding the fusion protein of the present invention and appropriate transcription/translation control signals. These methods include in vitro recombinant DNA technology, DNA synthesis technology, and in vivo recombination technology. The DNA sequence can be effectively linked to an appropriate promoter in the expression vector to guide mRNA synthesis. Representative examples of these promoters are: Escherichia coli lac or trp promoter; lambda phage PL promoter; eukaryotic promoters including CMV immediate early promoter, HSV thymidine kinase promoter, early and late SV40 promoter, retroviral LTRs and some other known promoters that can control gene expression in prokaryotic or eukaryotic cells or viruses. The expression vector also includes a ribosome binding site for translation initiation and a transcription terminator.

In addition, the expression vector preferably contains one or more selectable marker genes to provide phenotypic traits for selecting transformed host cells, such as dihydrofolate reductase for eukaryotic cell culture, neomycin resistance, and green fluorescent protein (GFP), or tetracycline or ampicillin resistance for E. coli.

A vector containing the above-mentioned appropriate DNA sequence and an appropriate promoter or control sequence can be used to transform an appropriate host cell so that it can express the protein.

The host cell can be a prokaryotic cell, such as Escherichia coli; or a lower eukaryotic cell, or a higher eukaryotic cell, such as a yeast cell, a plant cell or a mammalian cell (including a human and non-human mammal). Representative examples include: Escherichia coli, wheat germ cell, insect cell, SF9, Hela, HEK293, CHO, yeast cells, etc. In a preferred embodiment of the present invention, yeast cells (such as Pichia pastoris, Kluyveromyces, or a combination thereof; preferably, the yeast cells include: Kluyveromyces, more preferably Kluyveromyces marxianus, and/or Kluyveromyces lactis) are selected as host cells.

When the polynucleotide of the present invention is expressed in higher eukaryotic cells, if an enhancer sequence is inserted into the vector, the transcription will be enhanced. Enhancers are cis-acting factors of DNA, usually about 10 to 300 base pairs, acting on promoters to enhance gene transcription. Examples include the 100 to 270 base pair SV40 enhancer on the late side of the replication initiation point, the polyoma enhancer on the late side of the replication initiation point, and adenovirus enhancers and the like.

Those of ordinary skill in the art know how to select appropriate vectors, promoters, enhancers and host cells.

Transformation of host cells with recombinant DNA can be carried out by conventional techniques well known to those skilled in the art. When the host is a prokaryote such as Escherichia coli, competent cells that can absorb DNA can be harvested after the exponential growth phase and treated with the CaCl2 method. The steps used are well known in the art. Another method is to use MgCl2. If necessary, transformation can also be carried out by electroporation. When the host is a eukaryote, the following DNA transfection methods can be selected: calcium phosphate co-precipitation method, conventional mechanical methods such as microinjection, electroporation, liposome packaging, etc.

The obtained transformants can be cultured by conventional methods to express the polypeptide encoded by the gene of the present invention. Depending on the host cell used, the medium used in the culture can be selected from various conventional mediums. The culture is carried out under conditions suitable for the growth of the host cell. After the host cells have grown to an appropriate cell density, the selected promoter is induced by a suitable method (such as temperature conversion or chemical induction), and the cells are cultured for a period of time.

The recombinant polypeptide in the above method can be expressed in the cell or on the cell membrane, or secreted out of the cell. If necessary, using its physical, chemical and other characteristics to separate and purify the recombinant protein through various separation methods. These methods are well known to those skilled in the art. Examples of these methods include, but are not limited to: conventional renaturation treatment, treatment with protein precipitation agent (salting out method), centrifugation, bacteria broken through osmosis, ultra-treatment, ultra-centrifugation, molecular sieve chromatography (gel filtration), adsorption chromatography, ion exchange chromatography, high performance liquid chromatography (HPLC) and various other liquid chromatography techniques and combinations of these methods.

Gene Therapy

Gene Therapy for genetic diseases refers to the application of genetic engineering technology to introduce normal genes into patient cells to correct defective genes and cure the disease. The way of correction can be either to repair the defective gene in situ, or to transfer a functional normal gene into a certain part of the cell genome to replace the defective gene to play a role. Gene is the basic functional unit that carries biological genetic information, and is a specific sequence located on the chromosome. Certain technical methods or vectors must be used to introduce foreign genes into biological cells. The methods of gene transfer are divided into biological methods, physical methods and chemical methods. Adenovirus vectors are currently one of the most commonly used viral vectors for gene therapy. Gene therapy is mainly to treat those diseases that are serious threats to human health, including, but not limited to: genetic diseases (such as hemophilia, cystic fibrosis, family hypercholesterolemia, etc.), malignant tumors, cardiovascular diseases, infectious diseases (such as AIDS, rheumatoid, etc.). Gene therapy is a high-tech biomedical technology that introduces human normal genes or therapeutic genes into human target cells in a certain way to correct gene defects or exert therapeutic effects, so as to achieve the purpose of treating diseases. Gene therapy is different from conventional treatment methods: in general, the treatment of diseases is aimed at various symptoms caused by genetic abnormalities, while gene therapy is aimed at the causes of the disease—the abnormal gene itself. Target cells for gene therapy include, but are not limited to, somatic cells, bone marrow cells, liver cells, nerve cells, endothelial cells, and muscle cells.

In the present invention, the target gene is subjected to efficient gene editing (including gene insertion, replacement, etc.) through gene therapy, thereby restoring the normal expression of the gene or enhancing the expression of the gene, thereby treating related diseases.

The Main Advantages of the Present Invention Include:

(1) The present invention is first to demonstrate the fusion protein of the present invention can significantly improve gene editing efficiency in vivo or in vitro.
(2) The present invention is first to demonstrate the fusion protein of the present invention can significantly improve gene editing efficiency in vivo or in vitro. The increase rate is ≥20%, preferably, >40%, more preferably, >60% (such as 80%), up to 2 times.
(3) The present invention is first to demonstrate the in vitro transcription of mRNA of enhanced gene editing tool to improve the success of animal model construction.
(4) The present invention use AAV virus to package the enhanced gene editing tool, and express the protein of enhanced gene editing tools to improve the effect of disease treatment.
(5) The present invention is first to use the fusion of double-stranded DNA binding domain to improve the gene editing efficiency.
(6) The present invention is first to screen and find a double-stranded DNA binding domain (like HMG-D) that can efficiently improve gene editing efficiency, as well as its excellent fusion mode.
(7) This patent is first to discover that the double-stranded DNA binding domain can extensively improve gene editing efficiency of various gene editing tools.
(8) The enhanced gene editing tools in the present invention can improve the success rate of animal model construction, as well as gene therapy efficiency.
(9) The enhanced gene editing tools in the present invention also can apply to gene therapy.

The present invention will be further explained below in conjunction with specific embodiments. It should be understood that these embodiments are only used to illustrate the present invention and not to limit the scope of the present invention. The experimental methods without specific conditions in the following examples are usually based on conventional conditions, such as the conditions described in Sambrook et al., Molecular Cloning: Laboratory Manual (New York: Cold Spring Harbor Laboratory Press, 1989), or according to manufacturing The conditions suggested by the manufacturer. Unless otherwise specified, percentages and parts are weight percentages and parts by weight.

Unless otherwise specified, the reagents and materials in the examples of the present invention are all commercially available products.

General Methods:

### Method 1: (Applicable to Example 1, Example 2 and Example 3.

1. The density of HEK293T cells in a 24-well plate grows to 60-70%, and then an equimalor amount of plasmid was transfected into cells with transfection reagent PEI. The medium is changed for 8-10 hours, the cells were cultured for a period of time (Cas9 system cultured for 72 hours, base editing system cultured for 120 hours), harvesting the cells, and extracting the genome.
2. Design suitable primers to amplify a sequence of 150-180 bp around the target site, subjected to Hitom library construction, deep sequencing, analysis and calculation of editing efficiency.

### Method 2: (Applicable to Animal Model Construction in Example 4).

1. In vitro transcription of the mRNA of the enhanced gene editing tool and the sgRNA of the corresponding target.
2. mRNA and sgRNA were injected into mice embryos, then transferred into pseudopregnant mother mice to obtain FO generation mice, identify genes, count the mutation rate of mouse genotypes, and calculate the success rate of model construction.

### Method 3: (Applicable to Gene Therapy in Example 5).

1. Packaging adeno-associated virus (AAV) of enhanced gene editing tools and the sgRNA of the therapeutic gene, or the homologous repaired template.
2. Through intravenous injection (tail vein) or local injection (muscle, etc.) packaged AAV to the disease animal model (mouse or rat), and the control virus was also injected at the same time.
3. Regularly observation and test of the phenotype of the animal models in treatment group and control group to evaluate the efficiency of treatment.

### Example 1: Screening Enhanced Gene Editing Tools

To comparing the two endogenous targets (VEGF and HBG1/2), we synthesized various double-stranded DNA binding domain (HMG-D and Sac7d) respectively and designed five different lengths of linker (L1, L2, L3, L4 and L5) (Table 1), as well as fused to the N-terminus or C-terminus of Cas9. According to the resulting statistics, we scored the enhanced gene editing tools obtained from this series of optimizations (relative score based on the good results) (Bad: A; Not Good: AA; Better: AAA; Good: AAAA; Very Good: AAAAA), and the results are as follows: (note: C represents Cas9; L1-L5 represent various length linker; H represents HMG-D domain; S represents Sac7d)

### A: S-L1-C, H-L1-C

### AA: S-L2-C, S-L3-C, S-L4-C, H-L2-C

### AAA: H-L4-C-L4-S, H-L4-C-L5-S

### AAAA: H-L3-C, C-L4-S, C-L5-S, C-L4-H, H-L4-C-L4-H, H-L4-C-L5-H,

### H-L4-H-L4-C, C-L5-H-L5-H

### AAAAA: H-L4-C, C-L5-H

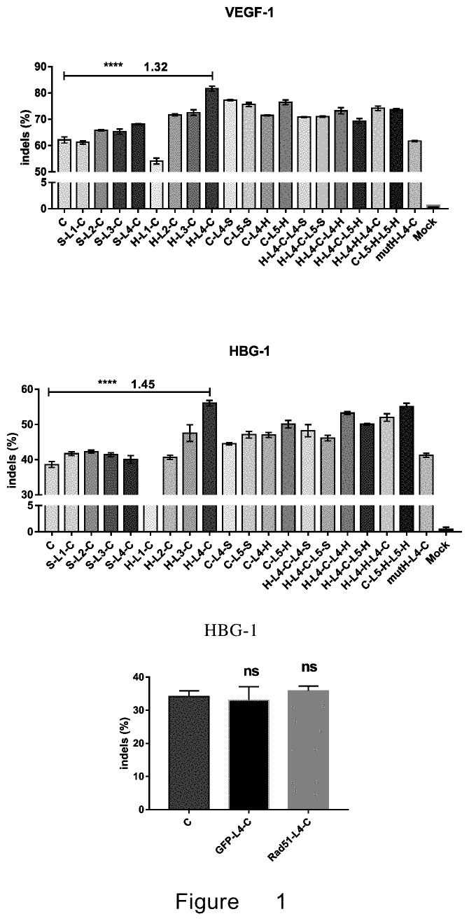

After comprehensive statistics, we have found HMG-D works very well, and the the linker of L4 and L5 length (32 and 64 amino acids) is the most optimal linker. Through comparing the N-terminal and C-terminal connections, we have found the effects of the N-terminal and C-terminal connections are both very good under the optimal linker condition. Therefore, the inventors have developed an enhanced gene editing tools, HMG-D is fused to the N-terminal or C-terminal of Cas9 through the L4 or L5 linker, which is a very good fusion method (FIG. 1).

Similarly, this enhanced gene editing tool, which enhances the efficiency of gene editing by fusing the double-stranded DNA binding domain, can also be applied to other types of double-stranded DNA binding domains, such as widely used zinc finger protein (ZFP), DNA binding domains of other transcription factors, and HMG-D or Sac7d from other species, etc. The present invention can also improve the efficiency of gene editing through these double-stranded DNA binding domains. Therefore, the most important thing of the present invention is that the double-stranded DNA binding domain fused gene editing tool can improve the efficiency of gene editing, and wherein the double-stranded DNA binding domain HMG-D is preferred.

### Example 2: Improving the Gene Editing Efficiency of SpCas9

The obtained enhanced gene editing tool (i.e., the fusion protein of the present invention, such as HMG-D-L4-SpCas9) is further compared in terms of effect on more endogenous targets. By transfecting 293T cells with an equimolar ratio, it is found that compared with SpCas9, the editing efficiency of the fusion protein of the present invention has been improved by more than 20% (or 60%, or 80%), up to 2 times (FIG. 2) in the compared targets.

### Example 3: Improving the Gene Editing Efficiency of Cas9 Protein Originated Other Species (Like SaCas9)

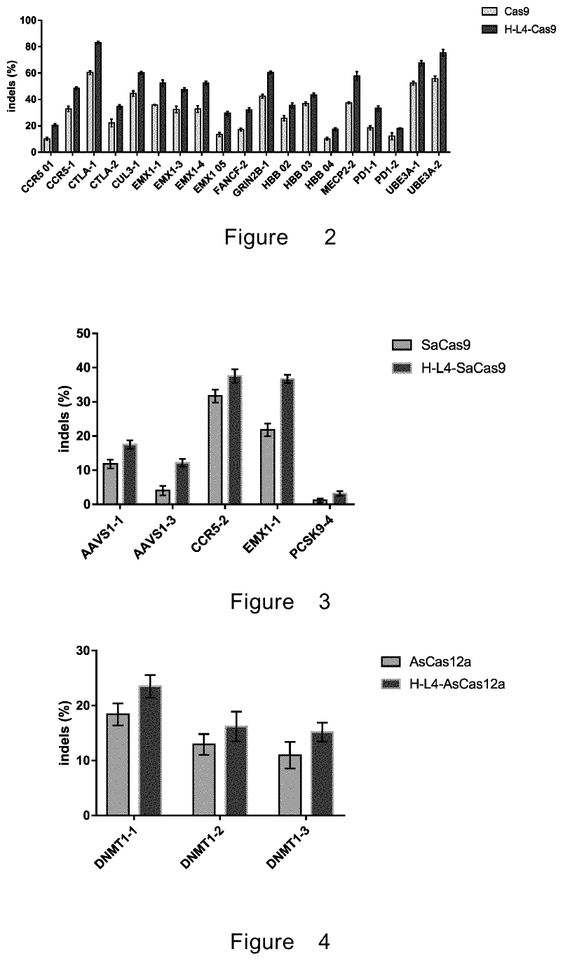

Trough constructing HMG-D-L4-SaCas9 expression vector and transfecting 293T cells at an equimolar ratio to target the endogenous target, we have found the editing efficiency of SaCas9 after HMG-D fusion also improves similarly (FIG. 3).

### Example 4: Improving the Gene Editing Efficiency of the Other Non-Cas9 Protein (Like AsCas12a)

Trough constructing HMG-D-L4-AsCas12a expression vector and transfecting 293T cells at an equimolar ratio to target the endogenous target, we have found the editing efficiency of AsCas12a after HMG-D fusion also improves similarly (FIG. 4).

### Example 5: Improving the Transcriptional Activation Efficiency of the Transcriptional Regulatory Tools (CRISPR/Cas9)

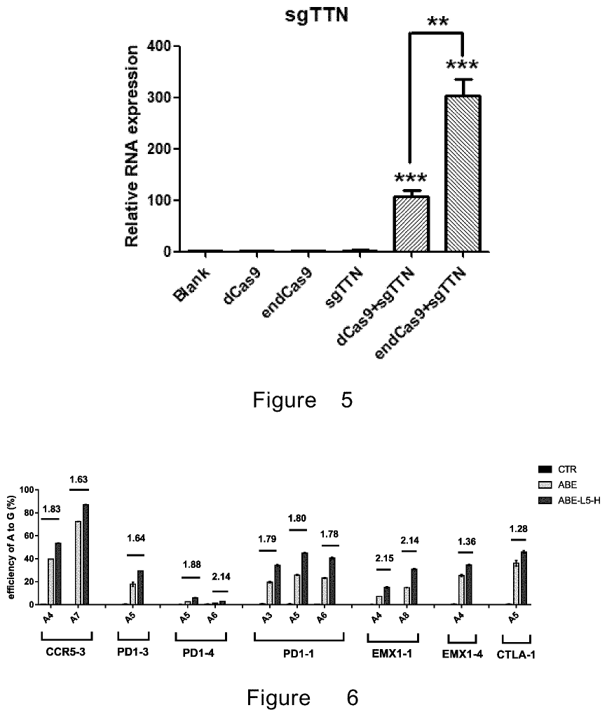

Trough constructing HMG-D-L4-dCas9-VPR expression vector and transfecting 293T cells at an equimolar ratio to target the endogenous target, we have found dCas9-VPR has a similar improvement effect on the transcriptional activation efficiency of endogenous genes after fusion of HMG-D. (FIG. 5).

### Example 6: Improving the Editing Efficiency of Base Editors

Since the base editor were developed by fusing cytosine deaminase (CBE) or adenine deaminase (ABE) to the N-terminal of Cas9, Therefore, at the C-terminal of Cas9 of the base editor, HMG-D was fused with the L5 linker to develop an enhanced single-base editing tool. Through the comparison of endogenous targets, we have found the editing efficiency of base editor (such as ABE) fused HMG-D have a huge improvement, such as by more than 1.5-2 times (FIG. 6). The improvement in editing efficiency of fusion proteins fused with other types of base editors is similar to or comparable to that of fusion proteins fused with ABE.

### Example 7: Improving the Success Rate of Animal Model Construction, and the Efficiency of Gene Therapy

Through in vitro transcription of mRNA of the enhanced gene editing tools, improving the success rate of animal model construction. Meanwhile, through packaging AAV virus of enhanced gene editing tools, the efficiency of disease treatment can also be improved.

### Comparative Example 1: Fusion of Non-DNA Binding Domain Show Poor Effect

To further validate the fusion of DNA binding domain can improve gene editing efficiency, we replaced DNA binding domain with the GFP protein (a non-DNA binding domain), an unrelated protein, and has found that the fusion of GFP can not improve gene editing efficiency (FIG. 1). Meanwhile, the HMG-D domain was mutated to destroy its DNA binding ability, and a 3 amino acid mutant HMG-D domain (mutHMG-D, referred to as mutH) was constructed. Through experimental comparison, the mutHMG-D domain can not improve the efficiency of gene editing (FIG. 1).

### Comparative Example 2: Apart from HMG-D and Sac7d, Other DNA Binding Domain have Poor Effect

To expand the scope of DNA binding domain, we also tested some single-stranded DNA binding domain (such as Rad51), and has found the fusion of Rad51 can not improve gene editing efficiency (FIG. 1). Hence, above results show the double-stranded DNA binding domain of HMG-D and Sac7d screened in the present invention are very effective.

All documents mentioned in the present invention are incorporated by reference herein as if each document were incorporated separately by reference. Furthermore, it should be understood that after reading the foregoing teachings of the invention, various changes or modifications may be made to the invention by those skilled in the art and that these equivalents are equally within the scope of the claims appended to this application.

1. A fusion protein, wherein the structure of the fusion protein is shown in the following Formula I or I′:
C-A-L-B (I)
B-L-A-C (I′)
wherein
A is a gene editing protein,
B is a DNA double-strand binding domain,
C is an optional base editor element;
L is none or a linker peptide; and wherein
each “-” is independently a linker peptide or a peptide bond or a non-peptide bond.

2. A polynucleotide, which encodes the fusion protein of claim 1.

3. The polynucleotide of claim 2 engineered into a vector.

4. The polynucleotide of claim 2 engineered into a host cell.

5-8. (canceled)

9. A pharmaceutical composition, comprising: (a) a fusion protein of claim 1, or a coding gene thereof, or an expression vector thereof; and (b) a pharmaceutically acceptable carrier.

10-11. (canceled)

12. A method for improving gene editing efficiency, comprising the steps: in the presence of the fusion protein of claim 1, performing gene editing on a cell, thereby improving the efficiency of gene editing.

13. The fusion protein of claim 1 wherein the gene editing protein (A) is selected from the group consisting of Cas9, Cas12, Cas12a, Cas12b, Cas13, Cas14, and a combination thereof.

14. The fusion protein of claim 1 wherein the gene editing protein (A) is selected from the group consisting of SEQ ID NO.: 1, SEQ ID NO.: 14 and SEQ ID NO.: 15.

15. The fusion protein of claim 1 wherein B is selected from HMG-D and Sac7d; and C is selected from adenine deaminase or cytosine deaminase or none; and L selected from L1, L2, L3, L4 and L5.

16. The fusion protein of claim 1 wherein B is HMG-D; and C is either adenine deaminase or cytosine deaminase or none; and L is L4 or L5.

17. The fusion protein of claim 1 wherein the fusion protein also comprises two or more of the A, B, C, and L elements in Formula I.

18. The fusion protein of claim 1 wherein B is HMG-9; and L is L4.

19. The fusion protein of claim 1 wherein B is HMG-D; C is adenine deaminase; and L is L5.

20. The fusion protein of claim 1 wherein B is HMG-D; C is cytosine deaminase; and L is L5.

21. The fusion protein of claim 1 wherein the amino acid sequence of the fusion protein of the present invention is as shown in SEQ ID NO.: 8, SEQ ID NO.: 9 or SEQ ID NO.: 13.

22. The fusion protein of claim 1, wherein the linker peptide is a sequence shown as Gly-Gly-Ser with n repeats, wherein n is 2-8, preferably n is 3-6.

23. The fusion protein of claim 1, wherein the DNA double-stranded binding domain is selected from the group consisting of HMG-D, Sac7d, and a combination thereof.

24. The fusion protein of claim 1, wherein the base editor element includes cytosine deaminase and adenine deaminase.


SEQUENCE LISTING (15 sequences)

SEQ ID NO: 1 - protein, 1423 aa, Streptococcus pyogenes
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 2 - protein, 1792 aa, artificial sequence
  base editor element
MPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 3 - protein, 9 aa, artificial sequence
  linker peptide
GGSGGSGGS

SEQ ID NO: 4 - protein, 18 aa, artificial sequence
  linker peptide
GGSGGSGGSGGSGGSGGS

SEQ ID NO: 5 - protein, 16 aa, artificial sequence
  linker peptide
SGSETPGTSESATPES

SEQ ID NO: 6 - protein, 32 aa, artificial sequence
  linker peptide
SGGSSGGSSGSETPGTSESATPESSGGSSGGS

SEQ ID NO: 7 - protein, 48 aa, artificial sequence
  linker peptide
SGGSSGGSSGSETPGTSESATPESSGSETPGTSESATPESSGGSSGGS

SEQ ID NO: 8 - protein, 1567 aa, artificial sequence
  fusion protein
MSDKPKRPLSAYMLWLNSARESIKRENPGIKVTEVAKRGGELWRAMKDKSEWEAKAAKAKDDYDRAVKEFEANGGSSAANGGGAKKRAKPAKKVAKKSKKEESDEDDDDESESGGSSGGSSGSETPGTSESATPESSGGSSGGSMDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 9 - protein, 1952 aa, artificial sequence
  fusion protein
MPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVSGGSSGGSSGSETPGTSESATPESSGSETPGTSESATPESSGGSSGGSMSDKPKRPLSAYMLWLNSARESIKRENPGIKVTEVAKRGGELWRAMKDKSEWEAKAAKAKDDYDRAVKEFEANGGSSAANGGGAKKRAKPAKKVAKKSKKEESDEDDDDESE

SEQ ID NO: 10 - protein, 112 aa, artificial sequence
  DNA double-strand binding domain
MSDKPKRPLSAYMLWLNSARESIKRENPGIKVTEVAKRGGELWRAMKDKSEWEAKAAKAKDDYDRAVKEFEANGGSSAANGGGAKKRAKPAKKVAKKSKKEESDEDDDDESE

SEQ ID NO: 11 - protein, 66 aa, artificial sequence
  DNA double-strand binding domain
MVKVKFKYKGEEKEVDTSKIKKVWRVGKMVSFTYDDNGKTGRGAVSEKDAPKELLDMLARAEREKK

SEQ ID NO: 12 - protein, 1667 aa, artificial sequence
  base editor element
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 13 - protein, 1827 aa, artificial sequence
  fusion protein
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVSGGSSGGSSGSETPGTSESATPESSGSETPGTSESATPESSGGSSGGSMSDKPKRPLSAYMLWLNSARESIKRENPGIKVTEVAKRGGELWRAMKDKSEWEAKAAKAKDDYDRAVKEFEANGGSSAANGGGAKKRAKPAKKVAKKSKKEESDEDDDDESE

SEQ ID NO: 14 - protein, 1057 aa, Staphylococcus aureus
MEASMKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 15 - protein, 1307 aa, Acidaminococcus sp
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN